(19) United States
(12) Patent Application Publication
(10) Pub. No.: US 2003/0059903 A1
(43) Pub. Date: Mar. 27, 2003

(54) Process for the production of L-amino acids using strains of the family enterobacteriaceae that contain an attenuated aceA gene

(75) Inventors: Mechthild Rieping, Bielefeld (DE); Thomas Hermann, Bielefeld (DE)

Correspondence Address: OBLON SPIVAK MCCLELLAND MAIER & NEUSTADT PC, FOURTH FLOOR, 1755 JEFFERSON DAVIS HIGHWAY, ARLINGTON, VA, 22202, US

(73) Assignee: DEGUSSA AG, Duesseldorf (DE)

(21) Appl. No.: 10/114,048

(22) Filed: Apr. 3, 2002

Related U.S. Application Data
(60) Provisional application No. 60/283,384, filed on Apr. 13, 2001.

(30) Foreign Application Priority Data
Apr. 3, 2001 (DE) 101 16 518.8

(51) Int. Cl. C12P 13/04; C12N 1/21

(52) U.S. Cl. 435/106; 435/252.3

(57) ABSTRACT

A process for the production of L-amino acids, in particular L-threonine, in which the following steps are carried out:

(a) fermentation of the microorganisms of the family Enterobacteriaceae producing the desired L-amino acid, in which the aceA gene or nucleotide sequences coding therefor are attenuated, in particular are switched off,

(b) enrichment of the L-amino acid in the medium or in the cells of the bacteria, and

(c) isolation of the L-amino acid.

### CROSS-REFERENCE TO RELATED APPLICATIONS

The present application claims priority to U.S. Provisional Application Serial No. 60/283,384, filed Apr. 13, 2001, the contents of which are incorporated herein by reference.

### FIELD OF THE INVENTION

The present invention relates to a process for the enzymatic production of L-amino acids, in particular L-threonine, using strains of the family Enterobacteriaceae in which the aceA gene is attenuated.

### DESCRIPTION OF THE BACKGROUND

L-amino acids, in particular L-threonine, are used in human medicine and in the pharmaceutical industry, in the foodstuffs industry, and most especially in animal nutrition. It is known to produce L-amino acids by fermentation of strains of Enterobacteriaceae, in particular Escherichia coli (E. coli) and Serratia marcescens. On account of their great importance efforts are constantly being made to improve processes for producing the latter. Process improvements may relate to fermentation technology measures, such as for example stirring and provision of oxygen, or the composition of the nutrient media, such as for example the sugar concentration during the fermentation, or the working-up to the product form, for example by ion exchange chromatography, or the intrinsic performance properties of the microorganism itself.

Methods comprising mutagenesis, selection and mutant choice are employed in order to improve the performance properties of these microorganisms. In this way strains are obtained that are resistant to antimetabolites, such as for example the threonine analogue a-amino-β-hydroxyvaleric acid (AHV) or are auxotrophic for regulatorily important metabolites, and that produce L-amino acids such as for example L-threonine.

Methods of recombinant DNA technology have also been used for some years in order to improve strains of the family Enterobacteriaceae producing L-amino acids, by amplifying individual amino acid biosynthesis genes and investigating their effect on production.

### SUMMARY OF THE INVENTION

The object of the invention is to provide new measures for the improved enzymatic production of L-amino acids, in particular L-threonine.

The present invention is based on the discovery microorganisms of the family Enterobacteriaceae which naturally produce L-amino acids do so more effectively under conditions in which the nucleotide sequence coding for the aceA gene is attenuated.

Thus, the object of the present invention may be accomplished with a process for the production of an L-amino acid, comprising:

(a) fermenting a microorganism of the family Enterobacteriaceae which produces the desired L-amino acid, in which the aceA gene or nucleotide sequences coding therefor are attenuated, in a medium;

(b) enriching the medium or the cells of the microorganism in the L-amino acid, and

(c) isolating the L-amino acid.

A more complete appreciation of the invention and many of the attendant advantages thereof will be readily obtained as the same becomes better understood by reference to FIG. 1 and the following detailed description.

### BRIEF DESCRIPTION OF THE DRAWINGS

FIG. 1: pMAK705ΔaceA (=pMAK705deltaaceA)

Length data are given as approximate values. The abbreviations and acronyms used have the following meanings:

| | |
|---|---|
| cat: | chloramphenicol resistance gene |
| rep-ts: | temperature-sensitive replication region of the plasmid pSC101 |
| ′aceB: | part of the 3′ region of the aceB gene |
| aceA′: | ATG start codon of the aceA gene |
| aceK′: | part of the 5′ region of the aceK gene |

The abbreviations for the restriction enzymes have the
following meanings:

| | |
|---|---|
| BamHI: | restriction endonuclease from Bacillus amyloliquefaciens |
| BglII: | restriction endonuclease from Bacillus globigii |
| ClaI: | restriction endonuclease from Caryphanon latum |
| EcoRI: | restriction endonuclease from Escherichia coli |
| EcoRV: | restriction endonuclease from Escherichia coli |
| HindIII: | restriction endonuclease from Haemophilus influenzae |
| KpnI: | restriction endonuclease from Klebsiella pneumoniae |
| PstI: | restriction endonuclease from Providencia stuartii |
| PvuI: | restriction endonuclease from Proteus vulgaris |
| SacI: | restriction endonuclease from Streptomyces |
| | achromogenes |
| SalI: | restriction endonuclease from Streptomyces albus |
| SmaI: | restriction endonuclease from Serratia marcescens |
| SphI: | restriction endonuclease from Streptomyces |
| | phaeochromogenes |
| SspI: | restriction endonuclease from Sphaerotilus species |
| XbaI: | restriction endonuclease from Xanthomonas badrii |
| XhoI: | restriction endonuclease from Xanthomonas holcicola |

### DETAILED DESCRIPTION OF THE INVENTION

Where L-amino acids or amino acids are mentioned hereinafter, this is understood to mean one or more amino acids including their salts, selected from the group comprising L-asparagine, L-threonine, L-serine, L-glutamate, L-glycine, L-alanine, L-cysteine, L-valine, L-methionine, L-isoleucine, L-leucine, L-tyrosine, L-phenylalanine, L-histidine, L-lysine, L-tryptophan and L-arginine. L-threonine is particularly preferred.

The term “attenuation” describes in this connection the reduction or switching off of the intracellular activity of one or more enzymes (proteins) in a microorganism that are coded by the corresponding DNA, by using for example a weak promoter or a gene or allele that codes for a corresponding enzyme with a low activity and/or that inactivates the corresponding enzyme (protein) or gene, and optionally combining these measures.

By means of these attenuation measures the activity or concentration of the corresponding protein is generally reduced to 0 to 75%, 0 to 50%, 0 to 25%, 0 to 10% or 0 to 5% of the activity or concentration of the wild type protein, or the activity or concentration of the protein in the initial microorganism.

The process is characterized in that the following steps are carried out:

(a) fermentation of microorganisms of the family Enterobacteriaceae in which the aceA gene is attenuated,

(b) enrichment of the corresponding L-amino acid in the medium or in the cells of the microorganisms of the family Enterobacteriaceae, and

(c) isolation of the desired L-amino acid, in which optionally constituents of the fermentation broth and/or the biomass in its entirety or portions thereof remain in the product.

The microorganisms that are the subject of the present invention can produce L-amino acids from glucose, sucrose, lactose, fructose, maltose, molasses, optionally starch, optionally cellulose or from glycerol and ethanol. The microorganisms are members of the family Enterobacteriaceae selected from the genera Escherichia, Erwinia, Providencia and Serratia. The genera Escherichia and Serratia are preferred. In the case of the genus Escherichia the species Escherichia coli may in particular be mentioned, and in the case of the genus Serratia the species Serratia marcescens may in particular be mentioned.

Suitable strains of the genus Escherichia, in particular those of the species Escherichia coli, that produce in particular L-threonine, include for example:

Escherichia coli TF427

Escherichia coli H4578

Escherichia coli KY10935

Escherichia coli VNIIgenetika MG442

Escherichia coli VNIIgenetika M1

Escherichia coli VNIIgenetika 472T23

Escherichia coli BKIIM B-3996

Escherichia coli kat 13

Escherichia coli KCCM-10132

Suitable strains of the genus Serratia, in particular of the species Serratia marcescens, that produce L-threonine include for example:

Serratia marcescens HNr21

Serratia marcescens TLr156

Serratia marcescens T2000

Strains of the family of Enterobacteriaceae producing L-threonine preferably have, inter alia, one or more of the genetic or phenotype features selected from the following group: resistance to a-amino-β-hydroxyvaleric acid, resistance to thialysine, resistance to ethionine, resistance to a-methylserine, resistance to diaminosuccinic acid, resistance to a-aminobutyric acid, resistance to borrelidin, resistance to rifampicin, resistance to valine analogues such as for example valine hydroxamate, resistance to purine analogues such as for example 6-dimethylaminopurine, need for L-methionine, optionally partial and compensable need for L-isoleucine, need for meso-diaminopimelic acid, auxotrophy with regard to threonine-containing dipeptides, resistance to L-threonine, resistance to L-homoserine, resistance to L-lysine, resistance to L-methionine, resistance to L-glutamic acid, resistance to L-aspartate, resistance to L-leucine, resistance to L-phenylalanine, resistance to L-serine, resistance to L-cysteine, resistance to L-valine, sensitivity to fluoropyruvate, defective threonine dehydrogenase, optionally ability to utilize sucrose, enhancement of the threonine operon, enhancement of homoserine dehydrogenase, I-aspartate kinase I, preferably of the feedback-resistant form, enhancement of homoserine kinase, enhancement of threonine synthase, enhancement of aspartate kinase, optionally of the feedback-resistant form, enhancement of aspartate semialdehyde dehydrogenase, enhancement of phosphoenol pyruvate carboxylase, optionally of the feedback-resistant form, enhancement of phosphoenol pyruvate synthase, enhancement of transhydrogenase, enhancement of the RhtB gene product, enhancement of the RhtC gene product, enhancement of the YfiK gene product, enhancement of a pyruvate carboxylase, and attenuation of acetic acid formation. It has now been found that microorganisms of the family Enterobacteriaceae after attenuation, in particular after switching off the aceA gene, produce L-amino acids, in particular L-threonine, in an improved way.

The nucleotide sequences of the Escherichia coli genes belong to the prior art and may also be obtained from the genome sequence of Escherichia coli published by Blattner et al. (Science 277, 1453-1462 (1997)).

The aceA gene is described inter alia by the following data:

| | |
|---|---|
| Designation: | Isocitrate lyase |
| EC-No.: | 4.1.3.1 |
| Reference: | Matsuoko and McFadden; Journal of |
| | Bacteriology 170, 4528-4536 (1988) |
| Accession No.: | AE000474 |

Apart from the described aceA gene, alleles of the gene may be used that result from the degeneracy of the genetic code or from functionally neutral sense mutations, the activity of the protein not being substantially altered.

In order to achieve an attenuation the expression of the gene or the catalytic properties of the enzyme proteins may for example be reduced or switched off. Optionally both measures may be combined.

The gene expression may be reduced by suitable culture conditions, by genetic alteration (mutation) of the signal structures of the gene expression, or also by antisense-RNA techniques. Signal structures of the gene expression are for example repressor genes, activator genes, operators, promoters, attenuators, ribosome-binding sites, the start codon and terminators. One person skilled in the art may find relevant information in, inter alia, articles by Jensen and Hammer (Biotechnology and Bioengineering 58: 191-195 (1998)), by Carrier and Keasling (Biotechnology Progress 15, 58-64 (1999), Franch and Gerdes (Current Opinion in Microbiology 3, 159-164 (2000)) and in known textbooks of genetics and molecular biology, such as for example the textbook by Knippers (“Molekulare Genetik”, 6^{th} Edition, Georg Thieme Verlag, Stuttgart, Germany, 1995) or that by Winnacker (“Gene and Klone”, VCH Verlagsgesellschaft, Weinheim, Germany, 1990).

Mutations that lead to a change or reduction of the catalytic properties of enzyme proteins are known from the prior art. As examples there may be mentioned the work by Qiu and Goodman (Journal of Biological Chemistry 272: 8611-8617 (1997)), Yano et al. (Proceedings of the National Academy of Sciences, USA 95, 5511-5515 (1998), Wente and Schachmann (Journal of Biological Chemistry 266, 20833-20839 (1991). Detailed information may be obtained from known textbooks on genetics and molecular biology, such as for example that by Hagemann (“Allgemeine Genetik”, Gustav Fischer Verlag, Stuttgart, 1986).

Suitable mutations include transitions, transversions, insertions and deletions. Depending on the action of the amino acid exchange on the enzyme activity, one speaks of missense mutations or nonsense mutations. Insertions or deletions of at least one base pair in a gene lead to frame shift mutations, which in turn lead to the incorporation of false amino acids or the premature termination of a translation. If as a result of the mutation a stop codon is formed in the coding region, this also leads to a premature termination of the translation. Deletions of several codons typically lead to a complete disruption of the enzyme activity. Details regarding the production of such mutations belong to the prior art and may be obtained from known textbooks on genetics and molecular biology, such as for example the textbook by Knippers (“Molekulare Genetik”, 6^{th} Edition, Georg Thieme Verlag, Stuttgart, Germany, 1995), that by Winnacker (“Gene und Klone”, VCH Verlagsgesellschaft, Weinheim, Germany, 1990) or that by Hagemann (“Allgemeine Genetik”, Gustav Fischer Verlag, Stuttgart, 1986).

Suitable mutations in the genes such as for example deletion mutations may be incorporated by gene and/or allele exchange in suitable strains.

A conventional method is the method of gene exchange by means of a conditionally replicating pSC101 derivate pMAK705 described by Hamilton et al. (Journal of Bacteriology 171, 4617-4622 (1989)). Other methods described in the prior art, such as for example that of Martinez-Morales et al. (Journal of Bacteriology 181, 7143-7148 (1999)) or that of Boyd et al. (Journal of Bacteriology 182, 842-847 (2000)) may likewise be used.

It is also possible to transfer mutations in the respective genes or mutations relating to the expression of the relevant genes, by conjugation or transduction into various strains.

Furthermore for the production of L-amino acids, in particular L-threonine, using strains of the family Enterobacteriaceae it may be advantageous in addition to the attenuation of the aceA gene also to enhance one or more enzymes of the known threonine biosynthesis pathway or enzymes of anaplerotic metabolism or enzymes for the production of reduced nicotinamide-adenine-dinucleotide phosphate.

The term “enhancement” describes in this connection the raising of the intracellular activity of one or more enzymes or proteins in a microorganism that are coded by the corresponding DNA, by for example increasing the number of copies of the gene or genes, using a strong promoter or a gene that codes for a corresponding enzyme or protein having a high activity, and optionally by combining these measures.

By means of the enhancement measures, in particular overexpression, the activity or concentration of the corresponding protein is in general raised by at least 10%, 25%, 50%, 75%, 100%, 150%, 200%, 300%, 400% or 500%, at most up to 1000% or 2000% referred to that of the wild type protein and/or the activity or concentration of the protein in the initial microorganism.

Thus, one or more of the genes selected from the following group may for example by simultaneously enhanced, in particular overexpressed: the thrABC operon coding for aspartate kinase, homoserine dehydrogenase, homoserine kinase and threonine synthase (U.S. Pat. No. 4,278,765),

the pyc gene coding for pyruvate carboxylase (DE-A-19 831 609), the pps gene coding for phosphoenol pyruvate synthase (Molecular and General Genetics 231:332 (1992)),

the ppc gene coding for phosphoenol pyruvate carboxylase (Gene 31:279-283 (1984)), the genes pntA and pntB coding for transhydrogenase (European Journal of Biochemistry 158:647-653 (1986)),

the gene rhtB imparting homoserine resistance (EP-A-0 994 190),

the mqo gene coding for malate:quinone oxidoreductase (DE 100 348 33.5),

the gene rhtC imparting threonine resistance (EP-A-1 013 765), and

the thrE gene of Corynebacterium glutamicum coding for threonine export (DE 100 264 94.8).

The use of endogenous genes is in general preferred. The term “endogenous genes” or “endogenous nucleotide sequences” is understood to mean the genes or nucleotide sequences present in the population of a species.

Furthermore for the production of L-amino acids, in particular L-threonine, it may be advantageous in addition to the attenuation of the aceA gene also to attenuate, in particular to switch off or reduce the expression of one or more of the genes selected from the following group:

the tdh gene coding for threonine dehydrogenase (Ravnikar and Somerville, Journal of Bacteriology 169, 4716-4721 (1987)),

the mdh gene coding for malate dehydrogenase (E.C. 1.1.1.37) (Vogel et al., Archives in Microbiology 149, 36-42 (1987)),

the gene product of the open reading frame (orf) yjfA (Accession Number AAC77180 of the National Center for Biotechnology Information (NCBI, Bethesda, Md., USA),

the gene product of the open reading frame (orf) ytfp (Accession Number AAC77179 des National Center for Biotechnology Information (NCBI, Bethesda, Md., USA),

the pckA gene coding for the enzyme phosphoenol pyruvate carboxykinase (Medina et al. (Journal of Bacteriology 172, 7151-7156 (1990)),

the poxB gene coding for pyruvate oxidase (Grabau and Cronan (Nucleic Acids Research 14 (13), 5449-5460 (1986)),

the dgsA gene coding for the regulator of the phosphotransferase system (Hosono et al., Bioscience, Biotechnology and Biochemistry 59, 256-251 (1995) and

Accession No.: AE000255), and

the fruR gene coding for the fructose repressor (Jahreis et al., Molecular and General Genetics 226, 332-336 (1991) and Accession No.: AE000118)

Furthermore for the production of L-amino acids, in particular L-threonine, it may be advantageous in addition to the attenuation of the aceA gene also to switch off undesirable secondary reactions (Nakayama: “Breeding of Amino Acid Producing Microorganisms”, in: Overproduction of Microbial Products, Krumphanzl, Sikyta, Vanek (eds.), Academic Press, London, UK, 1982).

The microorganisms produced according to the invention may be cultivated in a batch process (batch cultivation), in a fed batch process (feed process) or in a repeated fed batch process (repetitive feed process). A summary of known cultivation methods is described in the textbook by Chmiel (Bioprozesstechnik 1. Einführung in die Bioverfahrenstechnik (Gustav Fischer Verlag, Stuttgart, 1991)) or in the textbook by Storhas (Bioreaktoren and periphere Einrichtungen (Vieweg Verlag, Brunswick/Wiesbaden, 1994)).

The culture medium to be used must appropriately satisfy the requirements of the respective strains. Descriptions of culture media of various microorganisms are contained in the handbook “Manual of Methods for General Bacteriology” of the American Society for Bacteriology (Washington D.C., USA, 1981).

As carbon sources, sugars and carbohydrates such as for example glucose, sucrose, lactose, fructose, maltose, molasses, starch and optionally cellulose, oils and fats such as for example soya bean oil, sunflower oil, groundnut oil and coconut oil, fatty acids such as for example palmitic acid, stearic acid and linoleic acid, alcohols such as for example glycerol and ethanol, and organic acids such as for example acetic acid, may be used. These substances may be used individually or as a mixture.

As nitrogen source, organic nitrogen-containing compounds such as peptones, yeast extract, meat extract, malt extract, maize starch water, soya bean flour and urea or inorganic compounds such as ammonium sulfate, ammonium chloride, ammonium phosphate, ammonium carbonate and ammonium nitrate may be used. The nitrogen sources may be used individually or as a mixture. As phosphorus source, phosphoric acid, potassium dihydrogen phosphate or dipotassium hydrogen phosphate or the corresponding sodium-containing salts may be used. The culture medium must furthermore contain salts of metals, such as for example magnesium sulfate or iron sulfate, that are necessary for growth. Finally, essential growth promoters such as amino acids and vitamins may be used in addition to the aforementioned substances. Apart from these, suitable precursors may be added to the culture medium. The aforementioned starting substances may be added to the culture in the form of a single batch or may be metered in in an appropriate manner during the cultivation.

In order to regulate the pH of the culture basic compounds such as sodium hydroxide, potassium hydroxide, ammonia or ammonia water, or acidic compounds such as phosphoric acid or sulfuric acid are used as appropriate. In order to control foam formation antifoaming agents such as for example fatty acid polyglycol esters may be used. In order to maintain the stability of plasmids, suitable selectively acting substances, for example antibiotics, may be added to the medium. In order to maintain aerobic conditions, oxygen or oxygen-containing gas mixtures such as for example air are fed into the culture. The temperature of the culture is normally 25° C. to 45° C., and preferably 30° C. to 40° C. Cultivation is continued until a maximum amount of L-amino acids (or L-threonine) has been formed. This target is normally achieved within 10 hours to 160 hours.

The L-amino acids may be analyzed by anion exchange chromatography followed by ninhydrin derivation, as described by Spackman et al. (Analytical Chemistry, 30, (1958), 1190), or by reversed phase HPLC, as described by Lindroth et al. (Analytical Chemistry (1979) 51: 1167-1174).

The process according to the invention can be used for the enzymatic production of L-amino acids, such as for example L-threonine, L-isoleucine, L-valine, L-methionine, L-homoserine and L-lysine, in particular L-threonine.

A pure culture of the Escherichia coli K-12 strain DH5a/pMAK705 was filed as DSM 13720 on Sep. 8, 2000 at the German Collection for Microorganisms and Cell Cultures (DSMZ, Brunswick, Germany) according to the Budapest Convention.

The present invention is described in more detail hereinafter with the aid of examples of implementation.

The isolation of plasmid DNA from Escherichia coli as well as all techniques for the restriction, Klenow treatment and alkaline phosphatase treatment are carried out according to Sambrook et al. (Molecular Cloning—A Laboratory Manual (1989) Cold Spring Harbor Laboratory Press). The transformation of Escherichia coli is, unless otherwise described, carried out according to Chung et al. (Proceedings of the National Academy of Sciences of the United States of America, USA (1989) 86: 2172-2175).

The incubation temperature in the production of strains and transformants is 37° C. In the gene exchange process according to Hamilton et al., temperatures of 30° C. and 44° C. are used.

### EXAMPLES

Having generally described this invention, a further understanding can be obtained by reference to certain specific examples which are provided herein for purposes of illustration only and are not intended to be limiting unless otherwise specified. cl Example 1

### Construction of the Deletion Mutation of the aceA Gene

Parts of the gene regions lying upstream and downstream of the aceA gene are amplified from Escherichia coli K12 using the polymerase chain reaction (PCR) as well as synthetic oligonucleotides. Starting from the nucleotide sequence of the aceBAK operon in E. coli K12 MG1655 DNA (SEQ ID No. 1) the following PCR primers are synthesized (MWG Biotech, Ebersberg, Germany):

| | | |
|---|---|---|
| aceA′5′-1: | 5′-ATGCTTACTCACGCCTGTTG-3′ | (SEQ ID No.3) |
| aceA′5′-2: | 5′-CATGTGCAGATGCTCCATAG-3′ | (SEQ ID No.4) |
| aceA′3′-1: | 5′-CAACAACAACCGTTGCTGAC-3′ | (SEQ ID No.5) |
| aceA′3′-2: | 5′-CAGTTCGTTCGCCACCTGTA-3′ | (SEQ ID No.6) |

The chromosomal E. coli K12 MG1655 DNA used for the PCR is isolated according to the manufacturer's instructions using “Qiagen Genomic-tips 100/G” (QIAGEN, Hilden, Germany). A ca. 700 bp large DNA fragment from the region lying upstream of the aceA gene (designated ′aceB) and a ca. 800 bp large DNA fragment from the region lying downstream of the aceA gene (designated aceK') can be amplified with the specific primers under standard PCR conditions (Innis et al. (1990) PCR Protocols. A guide to methods and applications, Academic Press) with Taq DNA polymerase (Gibco-BRL, Eggenstein, Germany). The PCR products are ligated according to the manufacturer's instructions in each case with the vector pCR2. lTOPO (TOPO TA Cloning Kit, Invitrogen, Groningen, Netherlands) and transformed in the E. coli strain TOP 10F′. The selection of plasmid-carrying cells is carried out on LB agar to which 50 μg/ml of ampicillin has been added.

After the plasmid DNA isolation the vector pCR2.1 TOPO′ aceB is cleaved with the restriction enzymes EcoRV and SpeI, and the ′aceB fragment after separation in 0.8% agarose gel is isolated using the QIAquick Gel Extraction Kit (QIAGEN, Hilden, Germany). After the plasmid DNA isolation the vector pCR2.1TOPOaceK' is cleaved with the enzymes Ec11361I and SpeI and ligated with the isolated ′aceB fragment. The E. coli strain DH5a is transformed with the ligation batch and plasmid-carrying cells are selected on LB agar to which 50 μg/ml of ampicillin has been added. After the plasmid DNA isolation, those plasmids in which the mutagenic DNA sequence shown in SEQ ID No. 7 is present in cloned form are detected by control cleavage with the enzymes HindIII and XbaI. One of the plasmids is designated pCR2.1TOPOΔaceA.

### Example 2

### Construction of the Exchange Vector pMAK705ΔaceA

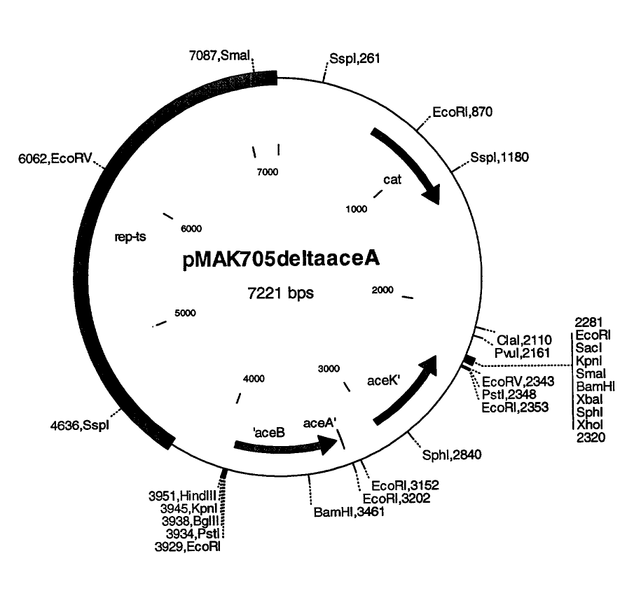

The aceBAK allele described in Example 1 is isolated from the vector pCR2.1TOPOΔaceA after restriction with the enzymes HindIIi and XbaI and separation in 0.8% agarose gel, and is ligated with the plasmid pMAK705 (Hamilton et al. (1989) Journal of Bacteriology 171, 4617-4622), that had been digested with the enzymes HindIII and XbaI. The ligation batch is transformed in DH5α and plasmid-carrying cells are selected on LB agar to which 20 μg/ml of chloramphenicol have been added. The successful cloning is detected after plasmid DNA isolation and cleavage with the enzymes BamHI, KpnI, SphI, SpeI and PstI. The resultant exchange vector pMAK705ΔaceA (=pMAK705deltaaceA) is shown in FIG. 1.

### Example 3

### Site-specific Mutagenesis of the aceA Gene in the E. coli Strain MG442

The E. coli

strain MG442 producing L-threonine is described in patent specification U.S. Pat. No. 4,278,765 and is filed as CMIM B-1628 at the Russian National Collection for Industrial Microorganisms (VKPM, Moscow, Russia).

For the exchange of the chromosomal aceA gene by the plasmid-coded deletion construct, MG442 is transformed with the plasmid pMAK705AaceA. The gene exchange is carried out by the selection process described by Hamilton et al. (1989) Journal of Bacteriology 171, 4617-4622) and is verified by standard PCR methods (Innis et al. (1990) PCR Protocols. A guide to methods and applications, Academic Press) with the following oligonucleotide primers:

| | | |
|---|---|---|
| aceA′5′-1: | 5′-ATGCTTACTCACGCCTGTTG-3′ | (SEQ ID No.3) |
| aceA′3′-2: | 5′-CAGTTCGTTCGCCACCTGTA-3′ | (SEQ ID No.6) |

### Example 4

### Production of L-threonine Using the Strain MG442ΔaceA

MG442AaceA is cultivated on minimal medium having the following composition: 3.5 g/l Na₂HPO₄.2H₂O, 1.5 g/l KH₂PO₄, 1 g/l NH₄Cl, 0.1 g/l MgSO₄.7H₂O, 2 g/l glucose and 20 g/l agar. The formation of L-threonine is checked in batch cultures of 10 ml that are contained in 100 ml Erlenmeyer flasks. For this, 10 ml of preculture medium of the following composition: 2 g/l yeast extract, 10 g/l (NH₄)₂SO₄, 1 g/l KH₂PO₄, 0.5 g/l MgSO₄.7H₂O, 15 g/l CaCO₃, 20 g/l glucose are inoculated and incubated for 16 hours at 37° C. and 180 rpm in an ESR incubator from Küthner AG (Birsfelden, Switzerland). 250 μl of this preculture are reinoculated in 10 ml of production medium (25 g/l (NH₄)₂SO₄, 2 g/l KH₂PO₄, 1 g/l MgSO₄.7H₂O, 0.03 g/l FeSO₄.7H₂O, 0.018 g/l MnSO₄.1H₂O, 30 g/l CaCO₃ and 20 g/l glucose) and incubated for 48 hours at 37° C. After incubation the optical density (OD) of the culture suspension is measured with an LP2W photometer from the Dr. Lange company (Dusseldorf, Germany) at a measurement wavelength of 660 nm.

The concentration of formed L-threonine is then determined in the sterile-filtered culture supernatant using an amino acid analyzer from Eppendorf-BioTronik (Hamburg, Germany) by ion exchange chromatography and post-column reaction with ninhydrin detection.

The result of the test is given in Table 1.

TABLE 1

| | OD | |
|---|---|---|
| Strain | (660 nm) | L-threonine g/l |
| MG442 | 6.0 | 1.5 |
| MG442ΔaceA | 6.2 | 1.9 |

The publications cited herein are incorporated herein by reference.

Obviously, numerous modifications and variations of the present invention are possible in light of the above teachings. It is therefore to be understood that within the scope of the appended claims, the invention may be practiced otherwise than as specifically described herein.

This application is based on German Patent Application Serial No. 101 16 518.8, filed on Apr. 3, 200 1, and incorporated herein by reference in its entirety.

What is claimed is:

1. A process for the production of an L-amino acid, comprising:
(a) fermenting a microorganism of the family Enterobacteriaceae which produces the desired L-amino acid, in which the aceA gene or nucleotide sequences coding therefor are attenuated, in a medium;
(b) enriching the medium or the cells of the microorganism in the L-amino acid, and
(c) isolating the L-amino acid.

2. The process of claim 1, wherein the L-amino acid is L-threonine.

3. The process of claim 1, wherein the aceA gene or nucleotide sequences coding therefor are switched off.

4. The process of claim 1, wherein constituents of the fermentation medium and/or the biomass in its entirety or portions thereof remain in the isolated L-amino acid.

5. The process of claim 1, wherein one or more genes in the biosynthesis pathway of the L-amino acid are enhanced in the microorganism.

6. The process of claim 1, wherein the metabolic pathways that reduce the formation of the L-amino acid are at least partially switched off in the microorganism.

7. The process of claim 1, wherein the expression of the aceA gene or nucleotide sequences coding therefor is attenuated.

8. The process of claim 1, wherein the expression of the aceA gene or nucleotide sequences coding therefor is switched off.

9. The process of claim 1, wherein the regulatory and/or catalytic properties of the polypeptide for which the aceA gene encodes are reduced.

10. The process of claim 1, wherein in the microorganism one or more of the genes selected from the following group is enhanced:
the thrABC operon coding for aspartate kinase, homoserine dehydrogenase, homoserine kinase and threonine synthase,
the pyc gene coding for pyruvate carboxylase,
the pps gene coding for phosphoenol pyruvate synthase,
the ppc gene coding for phosphoenol pyruvate carboxylase,
the pntA and pntB genes coding for transhydrogenase,
the rhtB gene imparting homoserine resistance,
the mqo gene coding for malate:quinone oxidoreductase,
the rhtC gene imparting threonine resistance, and
the thrE gene coding for threonine export.

11. The process of claim 1, wherein in the microorganism one or more of the genes selected from the following group is overexpressed:
the thrABC operon coding for aspartate kinase, homoserine dehydrogenase, homoserine kinase and threonine synthase,
the pyc gene coding for pyruvate carboxylase,
the pps gene coding for phosphoenol pyruvate synthase,
the ppc gene coding for phosphoenol pyruvate carboxylase,
the pntA and pntB genes coding for transhydrogenase,
the rhtB gene imparting homoserine resistance,
the mqo gene coding for malate:quinone oxidoreductase,
the rhtC gene imparting threonine resistance, and
the thrE gene coding for threonine export.

12. The process of claim 1, wherein in the microorganism one or more of the genes selected from the following group is attenuated:
the tdh gene coding for threonine dehydrogenase,
the mdh gene coding for malate dehydrogenase,
the gene product of the open reading frame (orf) yjfA,
the gene product of the open reading frame (orf) ytfp,
the pckA gene coding for phosphoenol pyruvate carboxykinase,
the poxB gene coding for pyruvate oxidase,
the dgsA gene coding for the regulator of the phosphotransferase system, and
the fruR gene coding for the fructose repressor.

13. The process of claim 1, wherein in the microorganism one or more of the genes selected from the following group is switched off:
the tdh gene coding for threonine dehydrogenase,
the mdh gene coding for malate dehydrogenase,
the gene product of the open reading frame (orf) yjfA,
the gene product of the open reading frame (orf) ytfp,
the pckA gene coding for phosphoenol pyruvate carboxykinase,
the poxB gene coding for pyruvate oxidase,
the dgsA gene coding for the regulator of the phosphotransferase system, and
the fruR gene coding for the fructose repressor.

14. The process of claim 1, wherein in the microorganism the expression of one or more of the genes selected from the following group is reduced:
the tdh gene coding for threonine dehydrogenase,
the mdh gene coding for malate dehydrogenase,
the gene product of the open reading frame (orf) yjfA,
the gene product of the open reading frame (orf) ytfp,
the pckA gene coding for phosphoenol pyruvate carboxykinase,
the poxB gene coding for pyruvate oxidase,
the dgsA gene coding for the regulator of the phosphotransferase system, and
the fruR gene coding for the fructose repressor.

15. The process of claim 1, wherein the microorganism belongs to the genus Escherichia.

16. The process of claim 1, wherein the microorganism belongs to the genus Erwinia.

17. The process of claim 1, wherein the microorganism belongs to the genus Providencia.

18. The process of claim 1, wherein the microorganism belongs to the genus Serratia.

19. The process of claim 1, wherein the microorganism is an E. coli.

20. The process of claim 1, wherein the microorganism is an Enterobacteriaceae selected from the group consisting of Escherichia coli MG442ΔaceA, Escherichia coli TF427, Escherichia coli, Escherichia coli KY10935, Escherichia coli VNIlgenetika MG442, Escherichia coli VNIIgenetika M1, Escherichia coli VNIIgenetika 472T23, Escherichia coli BKIIM B-3996, Escherichia coli kat 13, Escherichia coli KCCM-10132, Serratia marcescens HNr21, Serratia marcescens, and Serratia marcescens T2000.

21. The process of claim 1, wherein the L-amino acid is selected from the group consisting of L-asparagine, L-serine, L-glutamate, L-glycine, L-alanine, L-cysteine, L-valine, L-methionine, L-isoleucine, L-leucine, L-tyrosine, L-phenylalanine, L-histidine, L-lysine, L-tryptophan, and L-arginine.


SEQUENCE LISTING (7 sequences)

SEQ ID NO: 1 - DNA, 4855 bp, Escherichia coli
  CDS (1632)..(2936)
atgactgaacaggcaacaacaaccgatgaactggctttcacaaggccgtatggcgagcaggagaagcaaattcttactgccgaagcggtagaatttctgactgagctggtgacgcattttacgccacaacgcaataaacttctggcagcgcgcattcagcagcagcaagatattgataacggaacgttgcctgattttatttcggaaacagcttccattcgcgatgctgattggaaaattcgcgggattcctgcggacttagaagaccgccgcgtagagataactggcccggtagagcgcaagatggtgatcaacgcgctcaacgccaatgtgaaagtctttatggccgatttcgaagattcactggcaccagactggaacaaagtgatcgacgggcaaattaacctgcgtgatgcggttaacggcaccatcagttacaccaatgaagcaggcaaaatttaccagctcaagcccaatccagcggttttgatttgtcgggtacgcggtctgcacttgccggaaaaacatgtcacctggcgtggtgaggcaatccccggcagcctgtttgattttgcgctctatttcttccacaactatcaggcactgttggcaaagggcagtggtccctatttctatctgccgaaaacccagtcctggcaggaagcggcctggtggagcgaagtcttcagctatgcagaagatcgctttaatctgccgcgcggcaccatcaaggcgacgttgctgattgaaacgctgcccgccgtgttccagatggatgaaatccttcacgcgctgcgtgaccatattgttggtctgaactgcggtcgttgggattacatcttcagctatatcaaaacgttgaaaaactatcccgatcgcgtcctgccagacagacaggcagtgacgatggataaaccattcctgaatgcttactcacgcctgttgattaaaacctgccataaacgcggtgcttttgcgatgggcggcatggcggcgtttattccgagcaaagatgaagagcacaataaccaggtgctcaacaaagtaaaagcggataaatcgctggaagccaataacggtcacgatggcacatggatcgctcacccaggccttgcggacacggcaatggcggtattcaacgacattctcggctcccgtaaaaatcagcttgaagtgatgcgcgaacaagacgcgccgattactgccgatcagctgctggcaccttgtgatggtgaacgcaccgaagaaggtatgcgcgccaacattcgcgtggctgtgcagtacatcgaagcgtggatctctggcaacggctgtgtgccgatttatggcctgatggaagatgcggcgacggctgaaatttcccgtacctcgatctggcagtggatccatcatcaaaaaacgttgagcaatggcaaaccggtgaccaaagccttgttccgccagatgctgggcgaagagatgaaagtcattgccagcgaactgggcgaagaacgtttctcccaggggcgttttgacgatgccgcacgcttgatggaacagatcaccacttccgatgagttaattgatttcctgaccctgccaggctaccgcctgttagcgtaaaccaccacataactatggagcatctgcacatgaaaacccgtacacaacaaattgaagaattacagaaagagtggactcaaccgcgttgggaaggcattactcgcccatacagtgcggaagatgtggtgaaattacgcggttcagtcaatcctgaatgcacgctggcgcaactgggcgcagcgaaaatgtggcgtctgctgcacggtgagtcgaaaaaaggctacatcaacagcctcggcgcactgactggcggtcaggcgctgcaacaggcgaaagcgggtattgaagcagtctatctgtcgggatggcaggtagcggcggacgctaacctggcggccagcatgtatccggatcagtcgctctatccggcaaactcggtgccagctgtggtggagcggatcaacaacaccttccgtcgtgccgatcagatccaatggtccgcgggcattgagccgggcgatccgcgctatgtcgattacttcctgccgatcgttgccgatgcggaagccggttttggcggtgtcctgaatgcctttgaactgatgaaagcgatgattgaagccggtgcagcggcagttcacttcgaagatcagctggcgtcagtgaagaaatgcggtcacatgggcggcaaagttttagtgccaactcaggaagctattcagaaactggtcgcggcgcgtctggcagctgacgtgacgggcgttccaaccctgctggttgcccgtaccgatgctgatgcggcggatctgatcacctccgattgcgacccgtatgacagcgaatttattaccggcgagcgtaccagtgaaggcttcttccgtactcatgcgggcattgagcaagcgatcagccgtggcctggcgtatgcgccatatgctgacctggtctggtgtgaaacctccacgccggatctggaactggcgcgtcgctttgcacaagctatccacgcgaaatatccgggcaaactgctggcttataactgctcgccgtcgttcaactggcagaaaaacctcgacgacaaaactattgccagcttccagcagcagctgtcggatatgggctacaagttccagttcatcaccctggcaggtatccacagcatgtggttcaacatgtttgacctggcaaacgcctatgcccagggcgagggtatgaagcactacgttgagaaagtgcagcagccggaatttgccgccgcgaaagatggctataccttcgtatctcaccagcaggaagtgggtacaggttacttcgataaagtgacgactattattcagggcggcacgtcttcagtcaccgcgctgaccggctccactgaagaatcgcagttctaagcaacaacaaccgttgctgactgtaggccggataaggcgttcacgccgcatccggcaatcggtgcacgatgcctgatgcgacgcttgcgcgtcttatcatgcctacagccgttgccgaacgtaggctggataaggcgtttacgccgcatccggcaattctctgctcctgatgagggcgctaaatgccgcgtggcctggaattattgattgctcaaaccattttgcaaggcttcgatgctcagtatggtcgattcctcgaagtgacctccggtgcgcagcagcgtttcgaacaggccgactggcatgctgtccagcaggcgatgaaaaaccgtatccatctttacgatcatcacgttggtctggtcgtggagcaactgcgctgcattactaacggccaaagtacggacgcggcatttttactacgtgttaaagagcattacacccggctgttgccggattacccgcgcttcgagattgcggagagcttttttaactccgtgtactgtcggttatttgaccaccgctcgcttactcccgagcggctttttatctttagctctcagccagagcgccgctttcgtaccattccccgcccgctggcgaaagactttcaccccgatcacggctgggaatctctactgatgcgcgttatcagcgacctaccgctgcgcctgcgctggcagaataaaagccgtgacatccattacattattcgccatctgacggaaacgctggggacagacaacctcgcggaaagtcatttacaggtggcgaacgaactgttttaccgcaataaagccgcctggctggtaggcaaactgatcacaccttccggcacattgccatttttgctgccgatccaccagacggacgacggcgagttatttattgatacctgcctgacgacgaccgccgaagcgagcattgtttttggctttgcgcgttcttattttatggtttatgcgccgctgcccgcagcgctggtcgagtggctacgggaaattctgccaggtaaaaccaccgctgaattgtatatggctatcggctgccagaagcacgccaaaaccgaaagctaccgcgaatatctcgtttatctacagggctgtaatgagcagttcattgaagcgccgggtattcgtggaatggtgatgttggtgtttacgctgccgggctttgatcgggtattcaaagtcatcaaagacaggttcgcgccgcagaaagagatgtctgccgctcacgttcgtgcctgctatcaactggtgaaagagcacgatcgcgtgggccgaatggcggacacccaggagtttgaaaactttgtgctggagaagcggcatatttccccggcattaatggaattactgcttcaggaagcagcggaaaaaatcaccgatctcggcgaacaaattgtgattcgccatctttatattgagcggcggatggtgccgctcaatatctggctggaacaagtggaaggtcagcagttgcgcgacgccattgaagaatacggtaacgctattcgccagcttgccgctgctaacattttccctggcgacatgctgtttaaaaacttcggtgtcacccgtcacgggcgtgtggttttttatgattacgatgaaatttgctacatgacggaagtgaatttccgcgacatcccgccgccgcgctatccggaagacgaacttgccagcgaaccgtggtacagcgtctcgccgggcgatgttttcccggaagagtttcgccactggctatgcgccgacccgcgtattggtccgctgtttgaagagatgcacgccgacctgttccgcgctgattactggcgcgcactacaaaaccgcatacgtgaagggcatgtggaagatgtttatgcgtatcggcgcaggcaaagatttagcgtacggtatggggagatgcttttttga

SEQ ID NO: 2 - protein, 434 aa, Escherichia coli
MKTRTQQIEELQKEWTQPRWEGITRPYSAEDVVKLRGSVNPECTLAQLGAAKMWRLLHGESKKGYINSLGALTGGQALQQAKAGIEAVYLSGWQVAADANLAASMYPDQSLYPANSVPAVVERINNTFRRADQIQWSAGIEPGDPRYVDYFLPIVADAEAGFGGVLNAFELMKAMIEAGAAAVHFEDQLASVKKCGHMGGKVLVPTQEAIQKLVAARLAADVTGVPTLLVARTDADAADLITSDCDPYDSEFITGERTSEGFFRTHAGIEQAISRGLAYAPYADLVWCETSTPDLELARRFAQAIHAKYPGKLLAYNCSPSFNWQKNLDDKTIASFQQQLSDMGYKFQFITLAGIHSMWFNMFDLANAYAQGEGMKHYVEKVQQPEFAAAKDGYTFVSHQQEVGTGYFDKVTTIIQGGTSSVTALTGSTEESQF

SEQ ID NO: 3 - DNA, 20 bp, Artificial sequence
  Synthetic DNA
atgcttactcacgcctgttg

SEQ ID NO: 4 - DNA, 20 bp, Artificial sequence
  Synthetic DNA
catgtgcagatgctccatag

SEQ ID NO: 5 - DNA, 20 bp, Artificial sequence
  Synthetic DNA
caacaacaaccgttgctgac

SEQ ID NO: 6 - DNA, 20 bp, Artificial sequence
  Synthetic DNA
cagttcgttcgccacctgta

SEQ ID NO: 7 - DNA, 1643 bp, Escherichia coli
  misc_feature (1)..(33): Technical DNA/ remainder polylinker sequence
agcttggtaccgagatctgcagaattcgcccttatgcttactcacgcctgttgattaaaacctgccataaacgcggtgcttttgcgatgggcggcatggcggcgtttattccgagcaaagatgaagagcacaataaccaggtgctcaacaaagtaaaagcggataaatcgctggaagccaataacggtcacgatggcacatggatcgctcacccaggccttgcggacacggcaatggcggtattcaacgacattctcggctcccgtaaaaatcagcttgaagtgatgcgcgaacaagacgcgccgattactgccgatcagctgctggcaccttgtgatggtgaacgcaccgaagaaggtatgcgcgccaacattcgcgtggctgtgcagtacatcgaagcgtggatctctggcaacggctgtgtgccgatttatggcctgatggaagatgcggcgacggctgaaatttcccgtacctcgatctggcagtggatccatcatcaaaaaacgttgagcaatggcaaaccggtgaccaaagccttgttccgccagatgctgggcgaagagatgaaagtcattgccagcgaactgggcgaagaacgtttctcccaggggcgttttgacgatgccgcacgcttgatggaacagatcaccacttccgatgagttaattgatttcctgaccctgccaggctaccgcctgttagcgtaaaccaccacataactatggagcatctgcacatgaagggcgaattccagcacactggcggccgttactagtaacggccgccagtgtgctggaattcgcccttcaacaacaaccgttgctgactgtaggccggataaggcgttcacgccgcatccggcaatcggtgcacgatgcctgatgcgacgcttgcgcgtcttatcatgcctacagccgttgccgaacgtaggctggataaggcgtttacgccgcatccggcaattctctgctcctgatgagggcgctaaatgccgcgtggcctggaattattgattgctcaaaccattttgcaaggcttcgatgctcagtatggtcgattcctcgaagtgacctccggtgcgcagcagcgtttcgaacaggccgactggcatgctgtccagcaggcgatgaaaaaccgtatccatctttacgatcatcacgttggtctggtcgtggagcaactgcgctgcattactaacggccaaagtacggacgcggcatttttactacgtgttaaagagcattacacccggctgttgccggattacccgcgcttcgagattgcggagagcttttttaactccgtgtactgtcggttatttgaccaccgctcgcttactcccgagcggctttttatctttagctctcagccagagcgccgctttcgtaccattccccgcccgctggcgaaagactttcaccccgatcacggctgggaatctctactgatgcgcgttatcagcgacctaccgctgcgcctgcgctggcagaataaaagccgtgacatccattacattattcgccatctgacggaaacgctggggacagacaacctcgcggaaagtcatttacaggtggcgaacgaactgaagggcgaattctgcagatatccatcacactggcggccgctcgagcatgcat